(19) United States
(12) Patent Application Publication
(10) Pub. No.: US 2008/0253983 A1
(43) Pub. Date: Oct. 16, 2008

(54) SOS - Daily Control for Shiny and Oily Skin, Hair, Bald Scalp, and Body

(75) Inventor: Gayle Louise Williams, Fort Washington, MD (US)

Correspondence Address: GAYLE LOUISE WILLIAMS, 2009 THISTLEWOOD DRIVE, FORT WASHINGTON, MD, 20744, US

(73) Assignee: Gayle Louise Williams, Fort Washington, MD (US)

(21) Appl. No.: 12/032,658

(22) Filed: Apr. 14, 2008

Related U.S. Application Data
(60) Provisional application No. 61/028,174, filed on Feb. 12, 2008.

(51) Int. Cl. A61K 8/23; A61Q 99/00

(52) U.S. Cl. 424/70.1; 424/682

(57) ABSTRACT

When used as suggested, this invention uses Anhydrous MgSO4.7H2O as the primary substance, in liquid or solid forms, to control the appearance of shiny and oily skin, and hair; and also manages body moisture under normal situations and environmental conditions. Formulas are predetermined unit measurements to achieve or maintain the desired results.

I. Liquid forms are achieved by mixing the primary substance with water or other creams or liquids creating mild/gentle, medium/regular, and super/maximum strengths.

II. Solid forms can be mixed liquid or dry, then ground or compressed into a fine, compressed, or compact face or body powder.

III. Both forms can be added or combined with fragrance, skin creams, lotions, and cosmetic products.

IV. Either product can be reapplied as often as desired or needed.

### CROSS-REFERENCE TO RELATED APPLICATIONS

U.S. patent application Ser. No. 12/032,658

### STATEMENT REGARDING FEDERALLY SPONSORED RESEARCH OR DEVELOPMENT

There was no federally sponsored research or development associated with this Invention or patent application.

### THE NAMES OF THE PARTIES TO A JOINT RESEARCH AGREEMENT

There is no joint research agreement for this invention.

### REFERENCE TO A “SEQUENCE LISTING,” A TABLE, OR A COMPUTER PROGRAM LISTING APPENDIX SUBMITTED ON A COMPACT DISC AND AN INCORPORATION-BY-REFERENCE OF THE MATERIAL ON THE COMPACT DISC. THE TOTAL NUMBER OF COMPACT DISCS INCLUDING DUPLICATES AND THE FILES ON EACH COMPACT DISC SHALL BE SPECIFIED

There is no “Sequence Listing,” a table, or a computer program listing appendix submitted on a compact disc or incorporated-by-reference of material on the compact disc. The total number of compact discs including duplicates and the files on each compact disc shall be specified is zero.

1. This invention uses liquid or powdered forms of MgSO4.7H2O> to reduce and control the appearance of shiny and oily looking skin, hair, bald scalp areas, and body moisture when the proper form is directly applied to the skin or scalp, as desired. The skin or hair will look and feel dryer, less shiny, or oily, almost immediately after applying the invention solution substance and will remain in this temporary condition for several hours thereafter.
This substitute detailed description and specification of the invention, claim contains no new matter.
